(19) United States
(12) Patent Application Publication
(10) Pub. No.: US 2007/0059365 A1
(43) Pub. Date: Mar. 15, 2007

(54) Novel formulation of ropinirole

(76) Inventors: Peta Elizabeth Pollock, West Sussex (GB); Julian Westrup, Essex (GB); David Jonathan Yates, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/569,398

(22) PCT Filed: Aug. 19, 2004
(86) PCT No.: PCT/EP04/09356

§ 371 Date: Aug. 21, 2006

(30) Foreign Application Priority Data
Aug. 22, 2003 (GB) 0319874.4

(51) Int. Cl. A61K 31/404; A61K 9/22

(52) U.S. Cl. 424/468; 514/418

(57) ABSTRACT

The present invention relates to novel formulations of ropinirole for oral administration and to their use in the treatment of diseases which can prevent or disturb sleep, particularly Restless Legs Syndrome (RLS).

The present invention relates to novel formulations of ropinirole for oral administration and to their use in the treatment of diseases which can prevent or disturb sleep, particularly Restless Legs Syndrome (RLS).

Ropinirole hydrochloride (4-(2-di-n-propylaminoethyl)-2(3H)-indolone hydrochloride) is approved in most territories for the treatment of Parkinson's disease under the tradename ReQuip and has also been disclosed as being of potential use in the treatment of a variety of other conditions, such as Restless Legs Syndrome (RLS; Ekbom Newsletter, July 1997), fibromyalgia (U.S. Pat. No. 6,277,875), acute CNS injury (Medico, M. et al., (2002), European Neuropsychopharmacology 12, 187-194), various sleep related disorders such as apneas, hypopneas and snoring events (Saletu, M. et al., (2000), Neuropsychobiology 41, 190-199) and chronic fatigue syndrome (U.S. Pat. No. 6,300,365).

The present invention is particularly directed to an oral dosage formulation of ropinirole for the treatment of symptoms of diseases which can prevent or disturb sleep, such as Restless Legs Syndrome (RLS), apneas, hypopneas, snoring events, fibromyalgia and chronic fatigue syndrome, particularly RLS.

Ropinirole hydrochloride has previously only been disclosed as either an immediate release formulation or a 24-hour controlled release formulation (WO 01/78688). Since the half-life of ropinirole is approximately 5-6 hours, higher doses would be required to maintain therapeutic efficacy throughout the night when symptoms are present. Additionally, the 24-hour controlled release formulation may provide therapeutic concentrations of ropinirole during the daytime when symptoms are unlikely to be present.

Thus, for the treatment of RLS symptoms, there is a great need for a formulation of ropinirole with a release profile such that an RLS patient taking ropinirole in the early evening is provided with relatively rapidly relief of initial symptoms to allow onset of sleep (as indicated by a short duration to reach half peak plasma concentration (½Cmax) of ropinirole) followed by a sustained period wherein plasma concentration is maintained above ½Cmax to prevent RLS symptoms disturbing sleep. Ideally, concentrations of ropinirole should be negligible during the day when symptoms are unlikely to be present.

Thus, according to a first aspect of the present invention we provide a controlled release oral dosage form comprising a therapeutically effective amount of ropinirole or a salt thereof characterised in that:
the mean duration taken to achieve the half peak plasma concentration (½Cmax) of ropinirole in-vivo is less than 3 hours after administration of the oral dosage form; and
the mean duration above half peak plasma concentration (½Cmax) of ropinirole in-vivo is 7 to 13 hours.

‘Mean duration taken to achieve the half peak plasma concentration of ropinirole in-vivo’ refers to the average time to reach a plasma concentration of ropinirole equivalent to 50% of the maximum plasma concentration (Cmax) of ropinirole as measured in at least 8 human patients. Thus, the mean duration of time taken to attain half peak plasma concentration (½Cmax) provides an indication of likely onset of symptom relief.

Preferably, the mean duration taken to achieve the half peak plasma concentration (½Cmax) of ropinirole in-vivo is less than 2 hours after administration of the oral dosage form, more preferably between 1 and 2 hours.

‘Mean duration above half peak plasma concentration (½Cmax) of ropinirole in-vivo’ refers to the average time wherein plasma concentrations of ropinirole are maintained above half of the peak plasma concentration of ropinirole (½Cmax) as measured in at least 8 human patients. Thus, this value may be used as an indicator of duration of effect.

Preferably, the mean duration above half of the peak plasma concentration of ropinirole (½Cmax) is 7-12 hours.

Ropinirole, its chemical structure, processes for its preparation and therapeutic uses thereof, are more fully described in EP-A-0113964 (see Example 2), EP-A-0299602, EP-A-0300614, WO 91/16306, WO 92/00735 and WO 93/23035, and the contents of which are hereby incorporated by reference. “Ropinirole” as mentioned herein is defined as including pharmaceutically acceptable salts thereof. Most preferably, the ropinirole used in the dosage form is in the form of the hydrochloride salt. Ropinirole can be synthesised by the advantageous method described in WO 91/16306.

Thus, according to a second aspect of the present invention we provide a controlled release, oral dosage form comprising a therapeutically effective amount of ropinirole or a salt thereof, in a matrix wherein the in-vitro dissolution rate of the dosage form, when measured by the USP Paddle method at 50 rpm in 500 ml aqueous buffer (physiological pH range between 1 and 7) at 37° C. is:
between 20% and 55% (by weight) ropinirole released by 1 hour;
between 30% and 65% (by weight) ropinirole released by 2 hours;
between 70% and 95% (by weight) ropinirole released by 6 hours; and
greater than 80% (by weight) ropinirole released by 10 hours;
   the in-vitro release rate being independent of pH between pH 1 and 7.

USP Paddle Method is the Paddle Method described in US Pharmacopoeia, 26 (2003) using suitable sinkers to ensure that the dosage form does not adhere to the vessel.

The amounts released being, in all cases, a mean of at least 3 experiments.

Preferably, the dissolution rate is:
between 25% and 50% (by weight) ropinirole released by 1 hour;
between 45% and 65% (by weight) ropinirole released by 2 hours;
between 75% and 95% (by weight) ropinirole released by 6 hours; and
greater than 85% (by weight) ropinirole released by 10 hours.

More preferably, the dissolution rate is:
between 40% and 50% (by weight) ropinirole released by 1 hour;
between 60% and 70% (by weight) ropinirole released by 2 hours;
between 85% and 95% (by weight) ropinirole released by 6 hours; and
greater than 95% (by weight) ropinirole released by 10 hours.

Preferably, ropinirole hydrochloride is present within the oral dosage form at a concentration of between 0.05 and 10% (by weight of the dosage form), more preferably between 0.1 and 5%.

The oral dosage form according to the present invention is preferably presented as a tablet, granule, spheroid, bead, pellet or a capsule, more preferably a tablet.

The oral dosage form according to the present invention comprises any dosage form that affords the in-vitro dissolution rates within the ranges herein described and that which releases the ropinirole in a pH independent manner. Specific mention is made to U.S. Pat. No. 5,342,627 (specifically the control of drug release rate by manipulation of the geometry (and hence surface area) of the active substance dissolution core) the contents of which are herein incorporated by reference.

It will be appreciated that the oral dosage form of the present invention may comprise a monolith (e.g. a tablet comprising a homogenous mixture of all components) or a multi-component system (such as a multi-layer tablet (e.g. double layer tablet) or multi-particulate system) with different release rates from each component.

Preferably, the oral dosage form is a controlled release matrix comprising one or more dissolution rate controlling polymers in combination with one or more pharmaceutically acceptable excipients required to manufacture the final oral dosage form.

For example, when the oral dosage from is presented as a tablet, such excipients may comprise one or more diluents, binders, lubricants, glidants and/or disintegrants.

The dissolution rate controlling polymers function to manipulate the release rate of the drug. Suitable dissolution rate controlling polymers include, but are not limited to: cellulose ethers (e.g. hydroxypropylmethylcellulose (HPMC), ethylcellulose, hydroxypropylcellulose (HPC), hydroxyethylcellulose and carboxymethylcellulose sodium); polysaccharides (e.g. carageenan, guar gum, xanthan gum, tragacanth and ceratonia); polymethacrylates (e.g. copolymers of acrylic and methacrylic acid esters containing quaternary ammonium groups); cellulose esters (e.g. cellulose acetate); acrylic acid polymers (e.g. carbomers); waxes (e.g. hydrogenated castor oil, hydrogenated vegetable oil, carnauba wax and microcrystalline wax); alginates (e.g. alginic acid and sodium alginate); and fatty acid derivatives (e.g. glyceryl monostearate and glyceryl palmitostearate).

Preferably, the dissolution rate controlling polymers are selected from cellulose ethers, e.g. HPMC USP substitution types 1828, 2208, 2906 and 2910; ethylcellulose; HPC, weight average molecular weight 80,150,000, and xanthan gum, more preferably ethylcellulose and HPC or HPMC USP substitution types 2208 and 2910, especially HPMC USP substitution types 2208 and 2910.

When present, preferably one or more dissolution rate controlling polymers are contained within the dosage form such that the total concentration of dissolution rate controlling polymers ranges from 1 to 90% by weight of the dosage form, more preferably from 5 to 80%, especially from 30 to 40%.

Diluents may be present within the oral dosage form to increase tablet weight to an acceptable size for processing. Suitable diluents include, but are not limited to: calcium carbonate, calcium phosphate dibasic (anhydrous and dihydrate) and tribasic, microcrystalline cellulose, silicified microcrystalline cellulose, lactose (anhydrous and monohydrate), magnesium carbonate, maltitol, maltodextrin, maltose, mannitol, sorbitol and starch (e.g. pregelatinised starch).

Preferably, the diluents are selected from microcrystalline cellulose, lactose and mannitol, more preferably, microcrystalline cellulose and lactose (e.g. lactose monohydrate).

When present, preferably the diluents are contained within the dosage form in an amount ranging from 10% to 95% by weight of the dosage form, more preferably from 50 to 70%.

Binders may be present within the oral dosage form to aid the formation and maintain the integrity of granules. Suitable binders include, but are not limited to:

acacia, alginic acid, polyacrylic acids (e.g. carbomers), carboxymethylcellulose sodium, ceratonia, dextrin, ethylcellulose, HPMC, HPC, maltodextrin, polydextrose, polymethylmethacrylates and polyvinyl pyrrolidone (PVP).

Preferably, the binders are selected from PVP (weight average molecular weight 44,000-58,000), HPMC (USP substitution type 2910) and HPC (weight average molecular weight 80,000), more preferably HPMC (USP substitution type 2910) and HPC (weight average molecular weight 80,000), especially HPC (weight average molecular weight 80,000).

When present, preferably the binders are contained within the dosage form in an amount ranging from 0.5% to 10% by weight of the dosage form, more preferably 0.5% to 5%.

Lubricants may be present within the oral dosage form to prevent powder adhering to tablet punches during compression. Suitable lubricants include, but are not limited to: calcium stearate, glyceryl behenate, glyceryl monostearate, glyceryl palmitostearate, magnesium stearate, sodium benzoate, sodium stearyl fumarate, stearic acid, talc and zinc stearate.

Preferably, the lubricants are selected from stearates of magnesium, calcium and zinc, more preferably magnesium stearate.

When present, preferably the lubricants are contained within the dosage form in an amount ranging from 0.05 to 5% by weight of the dosage form, more preferably 0.1 to 1.5%, especially 0.5 to 1%.

Glidants may be present within the oral dosage form to improve powder flow during compression. Suitable glidants include, but are not limited to:

calcium phosphate tribasic, powdered cellulose, colloidal silicon dioxide, magnesium silicate, magnesium trisilicate and talc.

Preferably, the glidant is colloidal silicon dioxide.

When present, preferably the glidants are contained within the dosage form in an amount ranging from 0.1 to 5% by weight of the dosage form, more preferably 0.2 to 1.5%, especially 0.5%.

Disintegrants may be included in all or part of the oral dosage form to ensure rapid disintegration of the dosage form or part of the dosage from (for example, one of the layers in a double layer tablet) after administration. Suitable disintegrants include, but are not limited to:

alginic acid, carboxymethylcellulose calcium, carboxymethylcellulose sodium, croscarmellose sodium, crospovidone, guar gum, magnesium aluminium silicate, sodium alginate, sodium starch glycolate and starches.

Preferably, the disintegrants are selected from sodium starch glycolate and croscarmellose sodium, more preferably sodium starch glycolate.

When present, preferably the disintegrants are contained within the dosage form in an amount ranging from 0.1 to 15% by weight of the dosage form, more preferably 0.25 to 5%.

In addition to the above mentioned excipients, colour imparting substances may also be present within the oral dosage form to differentiate components within the formulation (e.g. different components in a multi-component system). Suitable colour imparting substances can be man-made dyes and lakes, or pigments derived from natural sources (or man-made counterparts of natural derivatives) that have been approved for use in drug products. Such materials include, but are not limited to, Beta-carotene, Brilliant Blue FCF (Food, Drug and Cosmetic (FD&C) Blue No. 1), Caramel, Cochineal extract (carmine/carminic acid), Indigotine (FD&C Blue No. 2, Indigo carmine), Iron oxides, synthetic (yellow ferric oxide, red ferric oxide and black ferric/ferrous oxide), Sunset Yellow FCF (FD&C Yellow No. 6), and Tartrazine (FD&C Yellow No.5).

Preferably, the colour imparting substance is ferric oxide, more preferably yellow ferric oxide.

When present, preferably the colour imparting substances are present within the dosage form in an amount ranging from 0.01 to 0.5% by weight of the dosage form, more preferably 0.02% to 0.2%, especially 0.025%.

When the oral dosage form of the present invention comprises a monolith, preferably the dosage form comprises one or more dissolution rate controlling polymers in combination with one or more diluents and one or more lubricants, optionally in combination with one or more binders and/or one or more glidants.

When the oral dosage form of the present invention comprises a double layer tablet, preferably the dosage form comprises one or more dissolution rate controlling polymers in combination with one or more diluents, one or more lubricants, one or more glidants and one or more colour imparting substances.

Preferably, the oral dosage form is a monolith comprising ropinirole hydrochloride, hydroxypropylmethylcellulose, lactose monohydrate and magnesium stearate.

Preferably, the oral dosage form is a double-layer tablet comprising ropinirole hydrochloride, hydroxypropylmethylcellulose, microcrystalline cellulose, sodium starch glycolate, magnesium stearate, colloidal silicon dioxide and yellow iron oxide.

Preferably, the oral dosage form is a monolith comprising ropinirole hydrochloride, hydroxypropylmethylcellulose, microcrystalline cellulose, colloidal silicon dioxide and magnesium stearate.

Preferably, the oral dosage form is a monolith comprising ropinirole hydrochloride, xanthan gum, lactose monohydrate and magnesium stearate.

Preferably, the oral dosage form is a monolith comprising ropinirole hydrochloride, hydroxypropylmethylcellulose, xanthan gum, microcrystalline cellulose, lactose monohydrate and magnesium stearate.

Preferably, the oral dosage form is a monolith comprising ropinirole hydrochloride, ethylcellulose, hydroxypropylcellulose, lactose monohydrate and magnesium stearate.

Preferably, the oral dosage form is a double-layer tablet comprising ropinirole hydrochloride, hydroxypropylmethylcellulose, microcrystalline cellulose, lactose monohydrate, colloidal silicon dioxide, magnesium stearate and yellow iron oxide.

Preferably, the oral dosage form is a monolith comprising ropinirole hydrochloride, hydroxypropylmethylcellulose, microcrystalline cellulose, lactose monohydrate, colloidal silicon dioxide and magnesium stearate.

Most preferably, the oral dosage form is a double-layer tablet comprising ropinirole hydrochloride, hydroxypropylmethylcellulose, microcrystalline cellulose, lactose monohydrate, colloidal silicon dioxide, magnesium stearate and yellow iron oxide.

Especially preferably, the oral dosage form is a double-layer tablet comprising in the first layer: 0.143 mg ropinirole hydrochloride, 20.756 mg microcrystalline cellulose, 10.376 mg lactose monohydrate and 5.625 mg HPMC; and in the second layer: 0.428 mg ropinirole hydrochloride, 45 mg HPMC, 43.594 mg microcrystalline cellulose and 21.791 mg lactose monohydrate.

Most especially preferably, the oral dosage form is a double-layer tablet comprising in the first layer: 0.143 mg ropinirole hydrochloride, 20.756 mg microcrystalline cellulose, 10.376 mg lactose monohydrate, 5.625 mg HPMC, 0.375 mg magnesium stearate and 0.188 mg colloidal silicon dioxide; and in the second layer: 0.428 mg ropinirole hydrochloride, 45 mg HPMC, 43.594 mg microcrystalline cellulose, 21.791 mg lactose monohydrate, 1.125 mg magnesium stearate and 0.563 mg colloidal silicon dioxide.

Preferably, the oral dosage form is a formulation as defined in any one of Examples 1-9, most preferably Example 8.

The dosage form of the present invention can be preferably prepared by compression of powder or granular mixtures, for example by blending followed by dry compression or wet granulation followed by compression, and preferably working between 1000 and 5000 kg/cm², employing procedures known to those skilled in the art.

In addition a covering may be applied to said finished tablets by a coating process and/or any other process well known to experts in the field.

The film coating may suitably comprise a polymer. Suitable polymers will be well known to the person skilled in the art and a non-limiting list of examples include cellulose ethers, for example hydroxypropylmethyl cellulose, hydroxypropyl cellulose or methylcellulose, and copolymers of methacrylic acid and methyl methacrylate. Preferably, the film coating will comprise hydroxypropylmethyl cellulose.

The total film coating solids are generally applied to the solid dosage form, for example the tablet core, in an amount of from 0.5 to 10% by weight, preferably about 1 to about 5%, more preferably about 2 to about 4% based on the dry weight of the dosage form. For example, about 6 mg of coat is applied to a tablet core weighing about 150 mg and about 9 mg of coat is applied to a tablet core weighing about 300 mg.

The film coating may additionally comprise any pharmaceutically acceptable colourants or opacifiers including water soluble dyes, aluminium lakes of water soluble dyes and inorganic pigments such as titanium dioxide and iron oxide.

The film coating may also contain one or more plasticising agents conventionally used in polymeric film coatings, for example, polyethylene glycol, propylene glycol, dibutyl sebecate, mineral oil, sesame oil, diethyl phthalate and triacetin. Proprietary film coating materials such as Opadry, obtainable from Colorcon Ltd., UK may be used.

A functional coat could also be applied to the tablet cores in order to modify the release rate of the active pharmaceutical ingredient. For example, application of a coat containing polymers insoluble at low pH's (e.g. copolymers of acrylic and methacrylic acid esters) will prevent drug being released in the acidic environment of the stomach. Application of a coat containing a polymer of low aqueous solubility (e.g. ethylcellulose) may be used to modify the overall rate of drug release.

It will be appreciated that the amount of ropinirole used within the dosage form according to the present invention will be such to result in the clinically determinable improvement in or suppression of symptoms of RLS. It will be understood, however, that the specific dose level for any particular patient will depend upon a variety of factors including the age, body weight, general health, sex, diet, time of administration, route of administration, rate of excretion, drug combination, and the severity of RLS. A suitable dosage unit of ropinirole for oral administration according to the present invention may comprise from 0.1 to 15 mg of ropinirole, preferably 0.25-10 mg. In order to ensure acceptable tolerability to the drug, the dosage should be titrated (using one or more dosage units, each of which could contain a different prescribed quantity of ropinirole) to achieve a maximal therapeutic effect.

The invention also provides a use of a dosage form as herein defined in the manufacture of a medicament for the treatment of diseases which can prevent or disturb sleep (particularly Restless Legs Syndrome).

The invention further provides a method of treatment of diseases which can prevent or disturb sleep (particularly Restless Legs Syndrome) that comprises administration of an oral dosage form as herein defined.

The following non-limiting examples illustrate the present invention:

### EXAMPLE 1 (E1)

Ropinirole hydrochloride (26.6 g) was high-shear mixed with lactose monohydrate (934 g). The blend was then low-shear mixed with lactose monohydrate (10069 g) and HPMC Methocel K4M (2791 g). Magnesium stearate (139.6 g) was then passed through a 1.0 mm screen and mixed into the blend.

A rotary tablet press was used to compress the blend into 46,667 tablet cores (target batch size) each containing:

| Ingredient | Function | % w/w | mg/tablet |
|---|---|---|---|
| ropinirole hydrochloride | active substance | 0.19 | 0.57 |
| HPMC (Methocel K4M; | dissolution rate | 20 | 60.00 |
| USP substitution type | controlling polymer | | |
| 2208; 4,000 mPa · s) | | | |
| lactose monohydrate | diluent | 78.81 | 236.43 |
| magnesium stearate | lubricant | 1 | 3.00 |

### EXAMPLE 2 (E2)

Blend ‘A’: Ropinirole hydrochloride (6.40 g) was high-shear mixed with microcrystalline cellulose (596.0 g), and yellow iron oxide (4.00 g). The blend was then low-shear mixed with microcrystalline cellulose (3221 g), and sodium starch glycolate (79.7 g). Magnesium stearate (39.84 g) and colloidal silicon dioxide (39.84 g) were then passed through a 1.0 mm screen and mixed into the blend.

Blend ‘B’: Ropinirole hydrochloride (16.4 g) was high-shear mixed microcrystalline cellulose (800.0 g). The blend was then low-shear mixed with microcrystalline cellulose (8128 g), and HPMC Methocel K4M (2662 g). Magnesium stearate (118.4 g) and colloidal silicon dioxide (118.4 g) were then passed through a 1.0 mm screen and mixed into the blend.

A rotary double layer press was used to compress blends A and B into 40,000 double layer tablet cores (target batch size) each containing:

| | | | |
|---|---|---|---|
| Component | Function | % w/w | mg/tablet |

Layer 1:

| | | | |
|---|---|---|---|
| ropinirole hydrochloride | active substance | 0.04 | 0.16 |
| microcrystalline | diluent | 23.935 | 95.74 |
| cellulose | | | |
| sodium starch glycolate | disintegrant | 0.5 | 2.00 |
| magnesium stearate | lubricant | 0.25 | 1.00 |
| colloidal silicon dioxide | glidant | 0.25 | 1.00 |
| yellow iron oxide | colour imparting | 0.025 | 0.1 |
| | substance | | |

Layer 2:

| | | | |
|---|---|---|---|
| ropinirole hydrochloride | active pharmaceutical | 0.1025 | 0.41 |
| | ingredient | | |
| hydroxypropylmethyl | dissolution rate | 16.875 | 67.50 |
| cellulose (Methocel | controlling polymer | | |
| K4M; USP substitution | | | |
| type 2208; 4,000 mPa · s) | | | |
| microcrystalline | diluent | 56.52 | 226.09 |
| cellulose | | | |
| magnesium stearate | lubricant | 0.75 | 3.00 |
| colloidal silicon dioxide | glidant | 0.75 | 3.00 |

After compression, tablets cores were coated with Opadry White OY-S-28876 to a target 3% w/w gain for cosmetic purposes.

### EXAMPLE 3 (E3)

Microcrystalline cellulose (136.749 g) and HPMC Methocel K15M (60.014 g) were blended by using a low-shear mixing process. Ropinirole hydrochloride (0.765 g) was then low-shear mixed with this blend by a process of trituration. Colloidal silicon dioxide (1.510 g) and magnesium stearate (1.002 g) were then passed through a 425 micron screen and mixed into the blend.

A single station tablet press was used to compress the blend into 1,333 tablet cores (target batch size) each containing:

| Ingredient | Function | % w/w | mg/tablet |
|---|---|---|---|
| ropinirole hydrochloride | active substance | 0.38 | 0.57 |
| HPMC (Methocel | dissolution rate | 30 | 45.00 |
| K15M; USP substitution | controlling polymer | | |
| type 2208, 15,000 | | | |
| mPa · s) | | | |
| microcrystalline | diluent | 68.37 | 102.56 |
| cellulose | | | |
| colloidal silicon dioxide | glidant | 0.75 | 1.13 |
| magnesium stearate | lubricant | 0.5 | 0.75 |

### EXAMPLE 4 (E4)

Ropinirole hydrochloride (0.57 g), lactose monohydrate (280.29 g) and xanthan gum Xantural (15.0 g) were combined and low-shear mixed for 5 minutes. Magnesium stearate (3.01 g) was then added and the blend mixed for a further 1 minute.

A single station tablet press was used to compress the blend into 1000 tablet cores (target batch size) each containing:

| Ingredient | Function | % w/w | mg/tablet |
|---|---|---|---|
| ropinirole hydrochloride | active substance | 0.19 | 0.57 |
| xanthan gum (Xantural) | dissolution rate | 5.02 | 15.06 |
| | controlling polymer | | |
| lactose monohydrate | diluent | 93.78 | 281.34 |
| magnesium stearate | lubricant | 1.01 | 3.03 |

### EXAMPLE 5 (E5)

Microcrystalline cellulose (91.567 g); lactose monohydrate (45.78 g); HPMC Methocel K100LV (56.005 g) and xanthan gum Xantural (3.997 g) were blended together using a low-shear mixing process. Ropinirole hydrochloride (0.671 g) was then low-shear mixed with this blend by a process of trituration. Magnesium stearate (2.006 g) was then passed through a 425 micron screen and mixed into the blend.

A single station tablet press was used to compress the blend into 1,333 cores (target batch size) each containing:

| Ingredient | Function | % w/w | mg/tablet |
|---|---|---|---|
| ropinirole hydrochloride | active substance | 0.33 | 0.50 |
| HPMC (Methocel K100LV; | dissolution rate | 28.00 | 42.00 |
| USP substitution type | controlling polymer | | |
| 2208; 100 mPa · s) | | | |
| xanthan gum (Xantural) | dissolution rate | 2.00 | 3.00 |
| | controlling polymer | | |
| microcrystalline | diluent | 45.78 | 68.67 |
| cellulose | | | |
| lactose monohydrate | diluent | 22.89 | 34.33 |
| magnesium stearate | lubricant | 1.00 | 1.50 |

### EXAMPLE 6 (E6)

Ropinirole hydrochloride (28.990 g) was high-shear mixed with lactose monohydrate (4271.1 g). The mix was then granulated with an aqueous solution of HPC Klucel EF (150 g) in purified water (550.309 g). The granules were then dried at 60° C. in a fluid bed dryer and subsequently passed through a 0.045 inch screen. The milled granules (3828.8 g) were then low-shear mixed with HPC Klucel LF, 450 microns (4510 g) and magnesium stearate (41.057 g).

The blend was compressed into 50,000 tablet cores (target batch size) using a single station tablet press fitted with specially designed tablet tooling such as those described in U.S. Pat. No. 5,342,627. Custom-designed fissures in the surface of the tablet cores were then filled with ethylcellulose (batch quantity 13,750 g) and the units compressed using a rotary tablet press to form tablets.

| Ingredient | Function | % w/w | mg/tablet |
|---|---|---|---|
| ropinirole hydrochloride | active substance | 0.12 | 0.58 |
| ethylcellulose | dissolution rate | 57.89 | 275.00 |
| | controlling polymer | | |
| hydroxypropylcellulose | dissolution rate | 23.16 | 110.00 |
| (Klucel LF; average | controlling polymer | | |
| molecular weight 95,000) | | | |
| lactose monohydrate | diluent | 17.98 | 85.42 |
| hydroxypropylcellulose | binder | 0.63 | 3.00 |
| (Klucel EF; average | | | |
| molecular weight | | | |
| 80,000) | | | |
| magnesium stearate | lubricant | 0.21 | 1.00 |

### EXAMPLE 7 (E7)

All ingredients were passed through a 900 micron screen prior to use.

Blend ‘A’: Ropinirole hydrochloride (61 g) was high-shear mixed with microcrystalline cellulose (2133 g) and yellow iron oxide (16.2 g). The blend was then low-shear mixed with microcrystalline cellulose (4968 g), HPMC Pharmacoat 603 (4655 g), lactose monohydrate (35189) and colloidal silicon dioxide (77.8 g). Magnesium stearate (155.29) was then mixed into the blend.

Blend ‘B’: Ropinirole hydrochloride (60.9 g) was high-shear mixed with microcrystalline cellulose (2133 g). The blend was then low-shear mixed with HPMC Methocel K15M (6207 g), microcrystalline cellulose (3944 g), lactose monohydrate (3006 g) and colloidal silicon dioxide (77.7 g). Magnesium stearate (155.2 g) was then mixed into the blend.

A rotary double layer press was used to compress blends A and B into 142,200 double layer tablet cores (target batch size) each containing:

| | | | |
|---|---|---|---|
| Component | Function | % w/w | mg/tablet |

Layer 1:

| | | | |
|---|---|---|---|
| ropinirole hydrochloride | active substance | 0.095 | 0.143 |
| microcrystalline cellulose | diluent | 11.337 | 17.006 |
| HPMC (Pharmacoat 603) | dissolution rate | 7.5 | 11.250 |
| | controlling polymer | | |
| lactose monohydrate | diluent | 5.667 | 8.501 |
| magnesium stearate | lubricant | 0.25 | 0.375 |
| colloidal silicon dioxide | glidant | 0.125 | 0.188 |
| yellow iron oxide | colour imparting | 0.025 | 0.038 |
| | substance | | |

Layer 2:

| | | | |
|---|---|---|---|
| ropinirole hydrochloride | active substance | 0.285 | 0.428 |
| HPMC (Methocel K15M) | dissolution rate | 30 | 45.000 |
| | controlling polymer | | |
| microcrystalline cellulose | diluent | 29.0627 | 43.594 |
| lactose monohydrate | diluent | 14.527 | 21.791 |
| magnesium stearate | lubricant | 0.75 | 1.125 |
| colloidal silicon dioxide | glidant | 0.375 | 0.563 |

After compression, tablet cores were coated with Opadry White OY-S-28876 to a target 4% w/w gain for cosmetic purposes.

### EXAMPLE 8 (E8)

All ingredients were passed through a 900 micron screen prior to use.

Blend ‘A’: Ropinirole hydrochloride (61 g) was high-shear mixed with microcrystalline cellulose (2133 g) and yellow iron oxide (16.2 g). The blend was then low-shear mixed with microcrystalline cellulose (6520 g), lactose monohydrate (4294 g), HPMC Pharmacoat 603 (2328 g) and colloidal silicon dioxide (77.8 g). Magnesium stearate (155.2 g) was then mixed into the blend.

Blend ‘B’: Ropinirole hydrochloride (60.9 g) was high-shear mixed with microcrystalline cellulose (2133 g). The blend was then low-shear mixed with HPMC Methocel K4M (6207 g), microcrystalline cellulose (3944 g), lactose monohydrate (30069) and colloidal silicon dioxide (77.7 g). Magnesium stearate (155.2 g) was then mixed into the blend.

A rotary double layer press was used to compress blends A and B into 142,200 double layer tablet cores (target batch size) each containing:

| | | | |
|---|---|---|---|
| Component | Function | % w/w | mg/tablet |

Layer 1:

| | | | |
|---|---|---|---|
| ropinirole hydrochloride | active substance | 0.095 | 0.143 |
| microcrystalline cellulose | diluent | 13.837 | 20.756 |
| lactose monohydrate | diluent | 6.917 | 10.376 |
| HPMC (Pharmacoat 603) | dissolution rate | 3.75 | 5.625 |
| | controlling polymer | | |
| magnesium stearate | lubricant | 0.25 | 0.375 |
| colloidal silicon dioxide | glidant | 0.125 | 0.188 |
| yellow iron oxide | colour imparting | 0.025 | 0.038 |
| | substance | | |

Layer 2:

| | | | |
|---|---|---|---|
| ropinirole hydrochloride | active substance | 0.285 | 0.428 |
| HPMC (Methocel K4M) | dissolution rate | 30 | 45.000 |
| | controlling polymer | | |
| microcrystalline cellulose | diluent | 29.0627 | 43.594 |
| lactose monohydrate | diluent | 14.527 | 21.791 |
| magnesium stearate | lubricant | 0.75 | 1.125 |
| colloidal silicon dioxide | glidant | 0.375 | 0.563 |

After compression, tablet cores were coated with Opadry White OY—S-28876 to a target 4% w/w gain for cosmetic purposes.

### EXAMPLE 9 (E9)

All ingredients were passed through a 900 micron screen prior to use.

Ropinirole hydrochloride (60.8 g) was high-shear mixed with microcrystalline cellulose (2133 g). The blend was then low-shear mixed with microcrystalline cellulose (4978 g), HPMC Methocel K4M (4655 g), lactose monohydrate (3524 g), and colloidal silicon dioxide (77.6 g). Magnesium stearate (155.2 g) was then mixed into the blend.

A rotary press was used to compress the blend into 106,667 tablet cores (target batch size) each containing:

| Component | Function | % w/w | mg/tablet |
|---|---|---|---|
| ropinirole hydrochloride | active substance | 0.38 | 0.57 |
| microcrystalline cellulose | diluent | 45.413 | 68.12 |
| HPMC (Methocel K4M) | dissolution rate | 30 | 45.00 |
| | controlling polymer | | |
| lactose monohydrate | diluent | 22.707 | 34.06 |
| magnesium stearate | lubricant | 1 | 1.50 |
| colloidal silicon dioxide | glidant | 0.5 | 0.75 |

After compression, tablet cores were coated with Opadry White OY—S-28876 to a target 4% w/w gain for cosmetic purposes.

### EXAMPLE 10

### In-Vitro Dissolution Studies with Examples 1-9 (E1-9)

In-vitro dissolution studies were conducted on tablets prepared in Examples 1-9. The dissolution method was the USP Paddle Method described in US Pharmacopoeia, 26 (2003). All studies were performed in 500 ml of aqueous buffer (pH4 Citrate Buffer) using a paddle speed of 50 rpm at a temperature of 37° C.

| | |
|---|---|
| Time | % weight of ropinirole hydrochloride released |

| (h) | E1 | E2 | E3 | E4 | E5 | E6 |
|---|---|---|---|---|---|---|
| 1 | 40 | 48 | 42 | 21 | 35 | 28 |
| 2 | 58 | 57 | 56 | 34 | 52 | 47 |
| 3 | — | — | — | — | — | 58 |
| 4 | 79 | 68 | 75 | 54 | 74 | — |
| 5 | — | — | — | — | — | 77 |
| 6 | 91 | 76 | 86 | 71 | 89 | — |
| 7 | — | — | — | — | — | 91 |
| 8 | 99 | 82 | 94 | 84 | 95 | — |
| 9 | — | — | — | — | — | 101 |
| 10 | 104 | 86 | 99 | 92 | 99 | 103 |

| Time | % weight of ropinirole hydrochloride released | | |
|---|---|---|---|
| (h) | E7 | E8 | E9 |
| 1 | 44 | 47 | 33 |
| 2 | 61 | 62 | 52 |
| 4 | 79 | 81 | 77 |
| 6 | 90 | 91 | 91 |
| 8 | 95 | 98 | 100 |
| 10 | 97 | 101 | 103 |
| 12 | 98 | 101 | 105 |

### EXAMPLE 11

### Pharmacokinetic Data for Examples 1, 2 and 6 (E1, E2 and E6)

Pharmcokinetic data for Examples 1, 2 and 6 (E1, E2 and E6) were generated in healthy volunteers during an open label study with a 4-way crossover, incomplete block design. Formulations were dosed in the morning as single doses in the fasted state with food and drink controlled and standardised. Each dosing session was separated by a 4 to 14 day washout period.

| | Mean Plasma Ropinirole Concentration (ng/ml) | | |
|---|---|---|---|
| Time (hours) | E1 (n = 8) | E2 (n = 9) | E6 (n = 9) |
| 0 | 0.000 ± 0.000 | 0.000 ± 0.000 | 0.000 ± 0.000 |
| 0.25 | 0.000 ± 0.000 | 0.022 ± 0.024 | 0.000 ± 0.000 |
| 0.5 | 0.027 ± 0.023 | 0.137 ± 0.092 | 0.031 ± 0.026 |
| 0.75 | 0.069 ± 0.049 | 0.269 ± 0.149 | 0.082 ± 0.058 |
| 1 | 0.117 ± 0.055 | 0.323 ± 0.185 | 0.121 ± 0.062 |
| 2 | 0.261 ± 0.109 | 0.392 ± 0.132 | 0.302 ± 0.112 |
| 3 | 0.337 ± 0.165 | 0.441 ± 0.191 | 0.412 ± 0.138 |
| 4 | 0.412 ± 0.191 | 0.456 ± 0.207 | 0.428 ± 0.139 |
| 6 | 0.430 ± 0.202 | 0.461 ± 0.290 | 0.436 ± 0.164 |
| 8 | 0.354 ± 0.148 | 0.377 ± 0.223 | 0.427 ± 0.160 |
| 10 | 0.259 ± 0.109 | 0.328 ± 0.184 | 0.377 ± 0.192 |
| 12 | 0.195 ± 0.099 | 0.274 ± 0.171 | 0.283 ± 0.162 |
| 14 | 0.143 ± 0.080 | 0.232 ± 0.151 | 0.226 ± 0.142 |
| 16 | 0.104 ± 0.049 | 0.207 ± 0.138 | 0.172 ± 0.117 |
| 18 | 0.085 ± 0.044 | 0.171 ± 0.111 | 0.142 ± 0.089 |
| 20 | 0.071 ± 0.036 | 0.142 ± 0.086 | 0.106 ± 0.070 |
| 22 | 0.052 ± 0.030 | 0.118 ± 0.072 | 0.089 ± 0.057 |
| 24 | 0.039 ± 0.022 | 0.099 ± 0.069 | 0.066 ± 0.045 |

Note:
n = number of volunteers dosed with the formulation

### EXAMPLE 12

### Pharmacokinetic Data for Examples 7-9 (E7-E9)

Pharmocokinetic data for Examples 7-9 (E7-E9) were generated in healthy volunteers during an open label crossover study. Formulations were dosed in the evening as single doses in the fed. Each dosing session was separated by a 4 to 14 day washout period.

| | Mean Plasma Ropinirole Concentration (ng/ml) | | |
|---|---|---|---|
| Time (hours) | E7 (n = 14) | E8 (n = 14) | E9 (n = 14) |
| 0 | 0.000 | 0.000 | 0.000 |
| 0.5 | 0.013 ± 0.024 | 0.036 ± 0.055 | 0.014 ± 0038 |
| 0.75 | 0.046 ± 0.060 | 0.096 ± 0.136 | 0.040 ± 0.074 |
| 1 | 0.094 ± 0.097 | 0.118 ± 0.125 | 0.061 ± 0.073 |
| 2 | 0.292 ± 0.141 | 0.289 ± 0.156 | 0.165 ± 0.117 |
| 3 | 0.345 ± 0.124 | 0.384 ± 0.144 | 0.264 ± 0.121 |
| 4 | 0.371 ± 0.131 | 0.419 ± 0.155 | 0.336 ± 0.096 |
| 6 | 0.326 ± 0.137 | 0.404 ± 0.159 | 0.377 ± 0.133 |
| 8 | 0.259 ± 0.118 | 0.297 ± 0.126 | 0.289 ± 0.128 |
| 10 | 0.208 ± 0.114 | 0.216 ± 0.106 | 0.226 ± 0.114 |
| 12 | 0.170 ± 0.118 | 0.186 ± 0.125 | 0.178 ± 0.093 |
| 14 | 0.137 ± 0.108 | 0.115 ± 0.077 | 0.142 ± 0.081 |
| 16 | 0.098 ± 0.093 | 0.086 ± 0.058 | 0.092 ± 0.068 |
| 24 | 0.029 ± 0.034 | 0.024 ± 0.023 | 0.026 ± 0.022 |

Note:
n = number of volunteers dosed with the formulation

Tradename Definitions

| Tradename | Generic Description | Supplier |
|---|---|---|
| Methocel | hydroxypropylmethylcellulose, USP | Dow |
| K4M | substitution type 2208, nominal | Chemical |
| | viscosity: 4,000 mPa · s for a | Company |
| | 2% w/w aqueous solution at 20° C. | |
| Methocel | hydroxypropylmethylcellulose, USP | Dow |
| K15M | substitution type 2208, nominal | Chemical |
| | viscosity: 15,000 mPa · s for a | Company |
| | 2% w/w aqueous solution at 20° C. | |
| Methocel | hydroxypropylmethylcellulose, USP | Dow |
| K100LV | substitution type 2208, nominal | Chemical |
| | viscosity: 100 mPa · s for a | Company |
| | 2% w/w aqueous solution at 20° C. | |
| Pharma- | hydroxypropylmethylcellulose, USP | Shin-Etsu |
| coat 603 | substitution type 2910, nominal | |
| | viscosity: 3 mPa · s for a | |
| | 2% w/w aqueous solution at 20° C. | |
| Xantural | Xanthan gum | CP Kelco |
| Klucel | Hydroxypropylcellulose, weight average | Aqualon |
| EF | molecular weight 80,000; aqueous solution | |
| | viscosity (typical 2% Brookfield): 7 mPa · s | |
| Klucel | Hydroxypropylcellulose, weight average | Aqualon |
| LF | molecular weight 95,000; aqueous solution | |
| | viscosity (typical 2% Brookfield): 10 mPa · s | |
| Opadry | hydroxypropylmethylcellulose aqueous | Colorcon |
| White | dispersion with polyethylene glycol | |
| (OY-S- | plasticizer and titanium dioxide pigment. | |
| 1-28876) | | |

What is claimed is:

1. A controlled release oral dosage form comprising a therapeutically effective amount of ropinirole or a salt thereof characterised in that:
the mean duration taken to achieve the half peak plasma concentration (½Cmax) of ropinirole in-vivo is less than 3 hours after administration of the oral dosage form; and
the mean duration above half peak plasma concentration (½Cmax) of ropinirole in-vivo is 7 to 13 hours.

2. A dosage form as defined in claim 1 wherein the mean duration taken to achieve the half peak plasma concentration (½Cmax) of ropinirole in-vivo is less than 2 hours after administration of the oral dosage form.

3. A dosage form as defined in claim 1 or claim 2 wherein the mean duration above half of the peak plasma concentration of ropinirole (½Cmax) is 7-12 hours.

4. A controlled release, oral dosage form comprising a therapeutically effective amount of ropinirole or a salt thereof, in a matrix wherein the in-vitro dissolution rate of the dosage form, when measured by the USP Paddle method at 50 rpm in 500 ml aqueous buffer (physiological pH range between 1 and 7) at 37° C. is:
between 20% and 55% (by weight) ropinirole released by 1 hour;
between 30% and 65% (by weight) ropinirole released by 2 hours;
between 70% and 95% (by weight) ropinirole released by 6 hours; and
greater than 80% (by weight) ropinirole released by 10 hours;
the in-vitro release rate being independent of pH between pH 1 and 7.

5. A dosage form as defined in claim 4 wherein said dissolution rate is:
between 25% and 50% (by weight) ropinirole released by 1 hour;
between 45% and 65% (by weight) ropinirole released by 2 hours;
between 75% and 95% (by weight) ropinirole released by 6 hours; and
greater than 85% (by weight) ropinirole released by 10 hours.

6. A dosage form as defined in claim 4 or claim 6 wherein said dissolution rate is:
between 40% and 50% (by weight) ropinirole released by 1 hour;
between 60% and 70% (by weight) ropinirole released by 2 hours;
between 85% and 95% (by weight) ropinirole released by 6 hours; and
greater than 95% (by weight) ropinirole released by 10 hours.

7. A dosage form as defined in claim 3 wherein ropinirole hydrochloride is present within the oral dosage form at a concentration of between 0.1 and 5% by weight of the dosage form.

8. A dosage form as defined in claim 3 which is presented as a tablet, granule, spheroid, bead, pellet or a capsule.

9. A dosage form as defined in claim 8 which is presented as a tablet.

10. A dosage form as defined in claim 8 which is a monolith or a double layer tablet.

11. A dosage form as defined in claim 3 which is a controlled release matrix comprising one or more dissolution rate controlling polymers in combination with one or more pharmaceutically acceptable excipients.

12. A dosage form as defined in claim 11 wherein said excipients comprise one or more diluents, binders, lubricants, glidants and/or disintegrants.

13. A dosage form as defined in claim 11 wherein said dissolution rate controlling polymers are selected from cellulose ethers, polysaccharides, polymethacrylates, cellulose esters, acrylic acid polymers, waxes, alginates and fatty acid derivatives.

14. A dosage form as defined in claim 12 wherein said diluents are selected from calcium carbonate, calcium phosphate dibasic and tribasic, microcrystalline cellulose, silicified microcrystalline cellulose, lactose, magnesium carbonate, maltitol, maltodextrin, maltose, mannitol, sorbitol and starch.

15. A dosage form as defined in claim 12 wherein said binders are selected from acacia, alginic acid, polyacrylic acids, carboxymethylcellulose sodium, ceratonia, dextrin, ethylcellulose, HPMC, HPC, maltodextrin, polydextrose, polymethylmethacrylates and polyvinyl pyrrolidone (PVP).

16. A dosage form as defined in claim 12 wherein said lubricants are selected from calcium stearate, glyceryl behenate, glyceryl monostearate, glyceryl palmitostearate, magnesium stearate, sodium benzoate, sodium stearyl fumarate, stearic acid, talc and zinc stearate.

17. A dosage form as defined in claim 12 wherein said glidants are selected from calcium phosphate tribasic, powdered cellulose, colloidal silicon dioxide, magnesium silicate, magnesium trisilicate and talc.

18. A dosage form as defined in claim 12 wherein said disintegrants are selected from alginic acid, carboxymethylcellulose calcium, carboxymethylcellulose sodium, croscarmellose sodium, crospovidone, guar gum, magnesium aluminium silicate, sodium alginate, sodium starch glycolate and starches.

19. A dosage form as defined in claim 10 which is a monolith comprising ropinirole hydrochloride, hydroxypropylmethylcellulose, lactose monohydrate and magnesium stearate.

20. A dosage form as defined in claim 10 which is a double layer tablet comprising ropinirole hydrochloride, hydroxypropylmethylcellulose, microcrystalline cellulose, sodium starch glycolate, magnesium stearate, colloidal silicon dioxide and yellow iron oxide.

21. A dosage form as defined in claim 10 which is a monolith comprising ropinirole hydrochloride, hydroxypropylmethylcellulose, microcrystalline cellulose, colloidal silicon dioxide and magnesium stearate.

22. A dosage form as defined in claim 10 which is a monolith comprising ropinirole hydrochloride, xanthan gum, lactose monohydrate and magnesium stearate.

23. A dosage form as defined in claim 10 which is a monolith comprising ropinirole hydrochloride, hydroxypropylmethylcellulose, xanthan gum, microcrystalline cellulose, lactose monohydrate and magnesium stearate.

24. A dosage form as defined in claim 10 which is a monolith comprising ropinirole hydrochloride, ethylcellulose, hydroxypropylcellulose, lactose monohydrate and magnesium stearate.

25. A dosage form as defined in claim 10 which is a double-layer tablet comprising ropinirole hydrochloride, hydroxypropylmethylcellulose, microcrystalline cellulose, lactose monohydrate, colloidal silicon dioxide and magnesium stearate.

26. A dosage form as defined in claim 10 which is a monolith comprising ropinirole hydrochloride, hydroxypropylmethylcellulose, microcrystalline cellulose, lactose monohydrate, colloidal silicon dioxide and magnesium stearate.

27. A dosage form as defined in claim 10 which is a double-layer tablet comprising ropinirole hydrochloride, hydroxypropylmethylcellulose, microcrystalline cellulose, lactose monohydrate, colloidal silicon dioxide and magnesium stearate.

28. A dosage form as defined in claim 10 which is a double-layer tablet comprising in the first layer: 0.143 mg ropinirole hydrochloride, 20.756 mg microcrystalline cellulose, 10.376 mg lactose monohydrate and 5.625 mg HPMC; and in the second layer: 0.428 mg ropinirole hydrochloride, 45 mg HPMC, 43.594 mg microcrystalline cellulose and 21.791 mg lactose monohydrate.

29. A dosage form as defined in claim 28 which is a double-layer tablet comprising in the first layer: 0.143 mg ropinirole hydrochloride, 20.756 mg microcrystalline cellulose, 10.376 mg lactose monohydrate, 5.625 mg HPMC, 0.375 mg magnesium stearate and 0.188 mg colloidal silicon dioxide; and in the second layer: 0.428 mg ropinirole hydrochloride, 45 mg HPMC, 43.594 mg microcrystalline cellulose, 21.791 mg lactose monohydrate, 1.125 mg magnesium stearate and 0.563 mg colloidal silicon dioxide.

30. (canceled)

31. (canceled)

32. A method of treatment of Restless Legs Syndrome which comprises administering to a host in need thereof an effective amount of an oral dosage form as defined in claim 10.

33. A pharmaceutical composition for use in the treatment of Restless Legs Syndrome which comprises an oral dosage form as defined in claim 10.
